(19) United States
(12) Patent Application Publication
(10) Pub. No.: US 2021/0252227 A1
(43) Pub. Date: Aug. 19, 2021

(54) CARTILAGE FILLING SYSTEM AND SURGICAL INSTRUMENT KIT INCLUDING THE SAME

(71) Applicant: BIOGEND THERAPEUTICS CO., LTD., Taipei City (TW)

(72) Inventors: Chin-Tsung HUANG, Taipei City (TW); Chun-Nan CHEN, Taipei City (TW); Po-Wei LEE, Taipei City (TW); Nai-Wen CHI, Taipei City (TW)

(21) Appl. No.: 17/002,242

(22) Filed: Aug. 25, 2020

(30) Foreign Application Priority Data
Feb. 17, 2020 (TW) 109201737

(51) Int. Cl. A61M 5/315; A61L 27/36; A61L 27/50; A61L 27/38; A61M 5/31

(52) U.S. Cl. CPC A61M 5/31511; A61L 27/3612; A61L 27/50; A61L 2430/06; A61M 5/3137; A61L 2400/06; A61L 27/3852

(57) ABSTRACT

The present invention provides a cartilage filling system and a surgical instrument kit including the same. The cartilage filling system comprises a filling push-rod, a filling member, a filling hopper, and a filtering adapter. The filling member comprises a syringe shaped as a hollow cylinder and comprising a first end portion and a second end portion, wherein the syringe has a first inner diameter; and a grip portion provided on the syringe. The filling hopper comprises a connecting member having an outer surface penetrated by a vent hole; and a hopper member comprising a wide portion and a narrow portion and having a second inner diameter, wherein the wide portion is connected to the connecting member, the narrow portion is detachably connected to the first end portion, and the second inner diameter is gradually reduced from the wide portion to the narrow portion, at which the second inner diameter is equal to the first inner diameter. The filtering adapter has an end connected to the second end portion and an opposite end connectable to an external syringe, wherein the filtering adapter comprises a vaned supporting portion and a filter membrane.

### BACKGROUND OF THE INVENTION

### 1. Technical Field

The present invention relates to a cartilage filling system and a surgical instrument kit including the same, and more particularly to a cartilage filling system and a surgical instrument kit including it that can simplify the surgical process.

### 2. Description of Related Art

The societies many of us live in are aging as the life expectancy of humans is increasing. To ensure the quality of life of the elderly, more and more attention has been paid to the treatment of various age-related diseases, including degenerative arthritis, which is a common degenerative disease. In the meantime, the growing prevalence of sports has increased the incidence of sports injuries associated with the articular cartilage. The treatment of degenerate or damaged joints, therefore, has been an important medical issue.

Cartilage scaffold implantation is often used to treat articular cartilage injuries. The conventional cartilage scaffold implantation procedure is nevertheless difficult because the surgeon is required to perform a complicated surgical operation in which each step calls for the use of specific instruments. As the instruments used in different steps of the operation are not designed to work with one another in a simple manner, it is necessary to transfer the cartilage tissue in use between different instruments in order to carry out such steps as filling and quantification. The transfers, however, add to the difficulty of cleaning those surgical instruments after use.

Therefore, a cartilage filling system and a surgical instrument kit that can simplify the conventional cartilage scaffold implantation procedure are needed.

### BRIEF SUMMARY OF THE INVENTION

In view of the aforesaid problems of the prior art, the present invention provides a cartilage filling system and a surgical instrument kit that can simplify the conventional cartilage scaffold implantation procedure.

The objective of the present invention is to provide a cartilage filling system comprising a filling push-rod, a filling member, a filling hopper, and a filtering adapter. The filling push-rod comprises a filling push-rod body and a filling push portion, wherein the filling push portion is provided at an end of the filling push-rod body. The filling member comprises a syringe shaped as a hollow cylinder and comprising a first end portion and a second end portion, wherein the syringe has a first inner diameter; and a grip portion provided on an outer surface of the syringe. The filling hopper comprises a connecting member having an outer surface with a plurality of projections, wherein the outer surface of the connecting member is penetrated by a vent hole; and a hopper member comprising a wide portion and a narrow portion, wherein the wide portion is connected to the connecting member, the wide portion has an inner diameter equal to an inner diameter of the connecting member, the narrow portion is detachably connected to the first end portion, the hopper member has a second inner diameter, and the second inner diameter is gradually reduced from the wide portion to the narrow portion, at which the second inner diameter is equal to the first inner diameter. The filtering adapter has an end connected to the second end portion and an opposite end connectable to an external syringe, wherein the filtering adapter comprises a vaned supporting portion and a filter membrane detachably provided on the vaned supporting portion.

Optionally, the cartilage filling system further comprises a pushing member and a quantifying carrier. The pushing member includes a bottom side; a sidewall surrounding the bottom side and connected to the bottom side such that the sidewall and the bottom side form a receiving space; and a rod member including a first rod portion and a second rod portion connected to each other, wherein the rod member penetrates the bottom side such that the first rod portion is in the receiving space while the second rod portion is outside the receiving space. The quantifying carrier includes a first connecting portion having an inner diameter corresponding to an outer diameter of the second end portion of the syringe; a second connecting portion having an inner diameter corresponding to an outer diameter of the sidewall of the pushing member; and a quantifying member shaped as a hollow cylinder and having an end connected to the first connecting portion and an opposite end connected to the second connecting portion; wherein the quantifying carrier is connected with the syringe through the first connecting portion and is connected with the pushing member through the second connecting portion.

Optionally, the first rod portion has a length less than a length of the second rod portion, and the quantifying member has a length greater than the length of the first rod portion and less than the length of the second rod portion.

Optionally, the quantifying member has an inner diameter which is the same as the first inner diameter, the first rod portion has an outer diameter greater than or equal to the first inner diameter, and the second rod portion has an outer diameter equal to the inner diameter of the quantifying member.

Optionally, the grip portion includes two ring-shaped gripping parts, each of the ring-shaped gripping parts is provided on the outer surface of the syringe, the grip portion is provided adjacent to the first end portion, and closed circular areas in the ring-shaped gripping parts lies in the same plane.

Optionally, the cartilage filling system further comprises a collection cup having an inner surface having structures corresponding in shape to the projections of the connecting member of the filling hopper to enable connection between the collection cup and the connecting member.

Another objective of the present invention is to provide a surgical instrument kit comprising the above cartilage filling system, a scaffold conveyor, a cartilage quantifying device, and a depth measuring device.

Optionally, the scaffold conveyor includes a conveying push-rod and a conveying member. The conveying member includes: a conveying tube having a conveying inner diameter and including a first conveying end and a second conveying end; a conveying wing plate provided on an outer surface of the conveying tube and adjacent to the first conveying end; and a grasping portion being a hollow circular base including a wide end and a narrow end, wherein the hollow circular base has a peripheral surface provided with a plurality of grooves that are arranged at intervals, the wide end is configured to connect with the second conveying end, and the wide end has an inner diameter equal to the conveying inner diameter. The conveying push-rod has an outer diameter corresponding to the conveying inner diameter so that at least a portion of the conveying push-rod can be inserted into the conveying tube, and the grasping portion has an outer diameter corresponding to the inner diameter of the first connecting portion.

Optionally, the cartilage quantifying device includes a quantifying push-rod, a quantifying base, and a quantifying member. The quantifying push-rod has a quantifying push-rod body and a quantifying plunger mounted around an end of the quantifying push-rod body. The quantifying base includes a plurality of supporting units and an upper connecting groove, wherein the supporting units are sequentially arranged around the upper connecting groove and about a center of circle defined by the center of the upper connecting groove, the supporting units are connected to one another, and each of the supporting units is connected to the upper connecting groove. The quantifying member includes: a hopper portion having a wide end and a narrow end; a quantifying tube shaped as a hollow cylinder, wherein the quantifying tube has a first quantifying end connected to the narrow end of the hopper portion and a second quantifying end detachably connected to the upper connecting groove, the quantifying tube has an inner diameter, the hopper portion has an inner diameter, and the inner diameter of the hopper portion is gradually reduced from the wide end to the narrow end, where the inner diameter of the hopper portion is equal to the inner diameter of the quantifying tube; and a quantifying grip portion provided on an outer surface of the quantifying tube. The quantifying plunger has an outer diameter corresponding to the inner diameter of the quantifying tube, and at least a portion of the quantifying push-rod is insertable into the quantifying tube such that a quantifying space is formed between a surface of the quantifying plunger, the quantifying tube, and the upper connecting groove.

Optionally, the second quantifying end of the quantifying tube has an engaging element, and an inner wall of the upper connecting groove has a coupling portion corresponding in shape to the engaging element to enable detachable connection between the second quantifying end of the quantifying tube and the upper connecting groove.

Optionally, the upper connecting groove has a bottom side penetrated by a plurality of vent holes, and the vent holes allow the air trapped by inserting at least a portion of the quantifying push-rod into the quantifying tube to flow out.

Optionally, the quantifying tube has graduation lines. When at least a portion of the quantifying push-rod is inserted into the quantifying tube and the surface of the quantifying plunger is aligned with one of the graduation lines, a quantifying space corresponding to the value of that graduation line is formed between the surface of the quantifying plunger, the quantifying tube, and the upper connecting groove.

Optionally, the depth measuring device is penetrated by a guide hole along an extending direction of the depth measuring device, and the depth measuring device includes: a guiding portion shaped as a rod; a positioning portion provided at a first end of the guiding portion and having a positioning outer diameter; and a measuring portion provided at a second end of the guiding portion and having a measuring outer diameter that is less than the positioning outer diameter.

The cartilage filling system of the present invention and the surgical instrument kit including it have the following advantages:

(1) The cartilage filling system of the present invention includes a pushing member and a quantifying carrier. It should be pointed out that the pushing member can be inserted into the quantifying carrier in a forward direction when used in conjunction with the filling member, in order for the first rod portion of the pushing member to form a specific receiving space, and that the pushing member can also be inserted into the quantifying carrier in the reverse direction when used in conjunction with the conveying member, in order for the second rod portion of the pushing member to push and thereby transfer the cartilage tissue in the quantifying carrier into the cavity of a scaffold. In other words, the pushing member, the quantifying carrier, the filling member, and the conveying member of the invention may have corresponding dimensions and shapes so that each one of them can be used in different ways to effectively reduce the number of the required surgical instruments. In addition, the filling hopper can reduce damage to the cartilage tissue in use, and the filter membrane in the filtering adapter can filter out unwanted liquids and impurities while retaining the cartilage tissue in the syringe.

(2) As the scaffold used in a cartilage scaffold implantation procedure must be able to receive and hold a specific amount of cartilage tissue, the cartilage quantifying device of the present invention can be used to quantify the required tissue precisely and thereby enhance the precision of the procedure. The cartilage quantifying device of the invention includes a quantifying member and a push-rod. By inserting the push-rod into the quantifying member and manipulating with the corresponding dimensions of the push-rod and the quantifying member as required by the scaffold, the required cartilage tissue can be quantified with precision.

(3) The surgical instrument kit of the present invention includes a scaffold conveyor and a depth measuring device in addition to the aforesaid cartilage filling system and cartilage quantifying device in order to facilitate the execution of each step of a cartilage scaffold implantation procedure. The components of the cartilage filling system and of the scaffold conveyor have matching diameters that make it easy to perform cartilage filling and scaffold conveyance; as a result, the process flow of the procedure is simplified, and the time required for cleaning the instruments after use is shortened. Moreover, as the depth measuring device is penetrated by a guide hole, the two ends of the depth measuring device can be used for positioning and for the measurement of depth respectively.

### BRIEF DESCRIPTION OF THE SEVERAL VIEWS OF THE DRAWINGS

FIG. 1 is a structural diagram of an embodiment of the cartilage filling system of the present invention.

FIG. 2 is a structural diagram of an embodiment of the filling push-rod of the present invention.

FIG. 3 shows structural diagrams of an embodiment of the filling member of the present invention.

FIG. 4 shows structural diagrams of an embodiment of the filling hopper of the present invention.

FIG. 5 shows structural diagrams of an embodiment of the filtering adapter of the present invention.

FIG. 6 shows structural diagrams of an embodiment of the pushing member of the present invention.

FIG. 7 shows structural diagrams of an embodiment of the quantifying carrier of the present invention.

FIG. 8 is a structural diagram of an embodiment of the scaffold conveyor of the present invention.

FIG. 9 is a structural diagram of an embodiment of the conveying push-rod of the present invention.

FIG. 10 is a structural diagram of an embodiment of the conveying member of the present invention.

FIG. 11 is a structural diagram of an embodiment of the cartilage quantifying device of the present invention.

FIG. 12 is a structural diagram of an embodiment of the quantifying push-rod of the present invention.

FIG. 13 shows structural diagrams of an embodiment of the quantifying plunger of the present invention.

FIG. 14 shows structural diagrams of an embodiment of the quantifying base of the present invention.

FIG. 15 is a structural diagram of an embodiment of the quantifying member of the present invention.

FIG. 16 is a structural diagram of an embodiment of the depth measuring device of the present invention.

FIG. 17 shows a drawing in which an embodiment of the pushing member of the present invention and an embodiment of the quantifying carrier of the invention are put together.

FIG. 18 shows a drawing in which an embodiment of the pushing member of the present invention and an embodiment of the quantifying carrier of the invention are shown in two different assembled states.

### DETAILED DESCRIPTION OF THE INVENTION

In order to facilitate the understanding of the technical features, content and advantages of the present invention and its achievable effects, the invention is described in detail in the form of expressions with the drawings and embodiments as follows. The main purpose of the drawings used is only to illustrate and assist in the description of the invention, and may not be the true proportions and precise configurations after the implementation of the invention. Therefore, the present invention should not be interpreted or limited to the proportions and configurations of the drawings. In addition, for ease of understanding, the same elements in the following embodiments are indicated by the same symbols.

Please note that unless otherwise specified or defined, the terms “connect” and “provide” as used in the following description of the present invention should be understood broadly. For example, the terms may refer to fixed connection, detachable connection, or integral connection, or refer to direct connection or indirect connection through an intermediate medium, or refer to communication between the interiors of two components. The aforesaid connections may be carried out through, for example but not limited to, engaging elements, screw threads, or magnetic attraction. A person of ordinary skill in the art should be able to understand the specific meanings of those terms according to the context in which they are used.

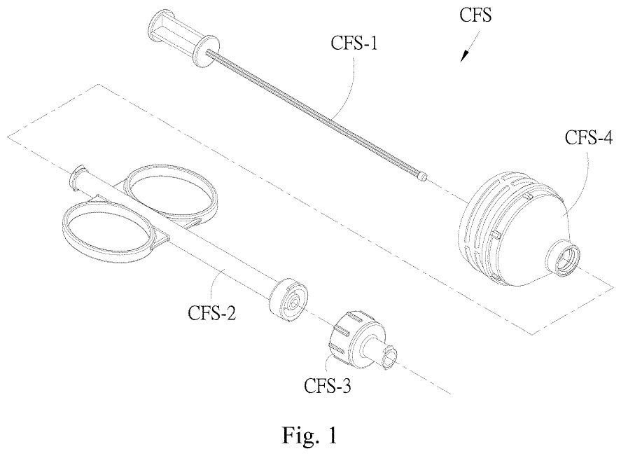

Refer to FIG. 1 for a structural diagram of an embodiment of the cartilage filling system of the present invention. The cartilage filling system CFS may include a filling push-rod CFS-1, a filling member CFS-2, a filtering adapter CFS-3, and a filling hopper CFS-4.

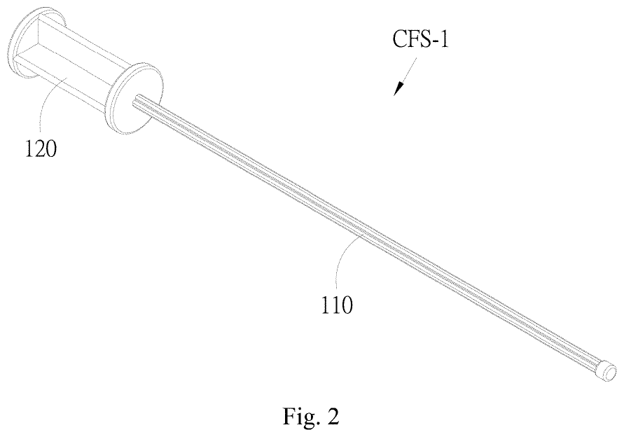

Refer to FIG. 2 for a structural diagram of an embodiment of the filling push-rod of the present invention. The filling push-rod CFS-1 may include a filling push-rod body 110 and a filling push portion 120. The filling push portion 120 may be provided at one end of the filling push-rod body 110. The filling push portion 120 may correspond in shape to a user's thumb so that a user can push the filling push portion 120 with ease.

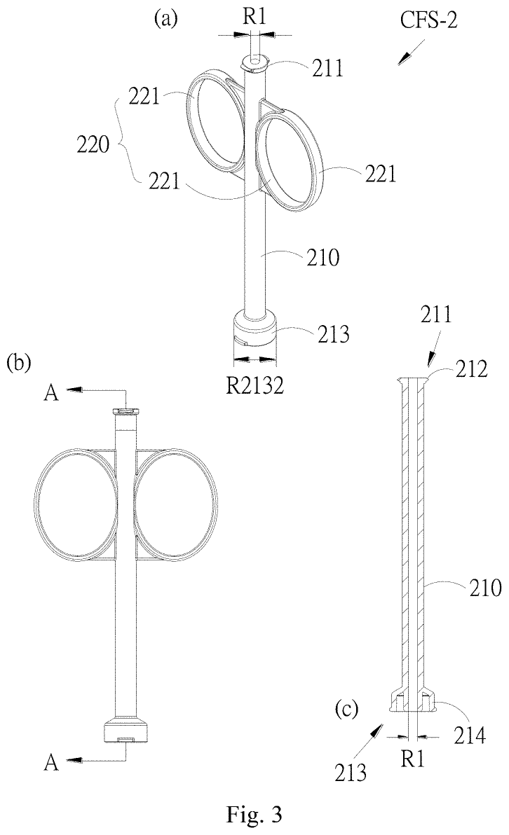

Refer to FIG. 3 for three structural diagrams of an embodiment of the filling member of the present invention, in which FIG. 3(a) to FIG. 3(c) are respectively a perspective view, a front view, and a line A-A sectional view of the filling member of the invention. The filling member CFS-2 may include a syringe 210 and a grip portion 220. The syringe 210 may be shaped as a hollow cylinder or a hollow column with an arbitrary cross-sectional shape, e.g., a hollow pentagonal or hexagonal prism as appropriate. The syringe 210 may include a first end portion 211 and a second end portion 213. The syringe 210 may have a first inner diameter R1. The first inner diameter R1 is preferably 1.9 mm˜3.1 mm, more preferably 2.1 mm˜2.9 mm, and even more preferably 2.3 mm˜2.7 mm. The second end portion 213 may have an outer diameter R2132. The outer diameter R2132 is preferably 11.8 mm˜13.2 mm, more preferably 12 mm˜13 mm, and even more preferably 12.2 mm˜12.8 mm.

The outer diameter of the filling push-rod body 110 of the filling push-rod CFS-1 matches the first inner diameter R1 so that the filling push-rod body 110 can be inserted smoothly into the syringe 210 without creating a large number of gaps between the filling push-rod body 110 and the inner wall of the syringe 210.

The grip portion 220 may be provided on the outer surface of the syringe 210 and is configured to be gripped by a user. The grip portion 220 may be shaped as a hollow circular plate or a plurality of hollow plates symmetrical to an axis of symmetry defined by the syringe 210. In one embodiment as shown in FIG. 3(a), the grip portion 220 may have two or more ring-shaped gripping parts 221, and each of the ring-shaped gripping parts 221 is provided on the outer surface of the syringe 210. The grip portion 220 is provided adjacent to the first end portion 211. The closed circular areas in the ring-shaped gripping parts 221 may lie in the same plane; that is to say, the ring-shaped gripping parts 221 may be provided in the same plane to facilitate gripping.

Referring to the sectional view of FIG. 3(c), the syringe 210 may have the first inner diameter R1; the first end portion 211 of the syringe 210 may have a protuberance 212 provided on the outer surface of the first end portion 211, wherein the protuberance 212 is configured to connect with the filling hopper CFS-4; and the second end portion 213 of the syringe 210 may have a pair of split ends 214 provided on the outer surface of the second end portion 213, wherein the split ends 214 are configured to connect with the filtering adapter CFS-3.

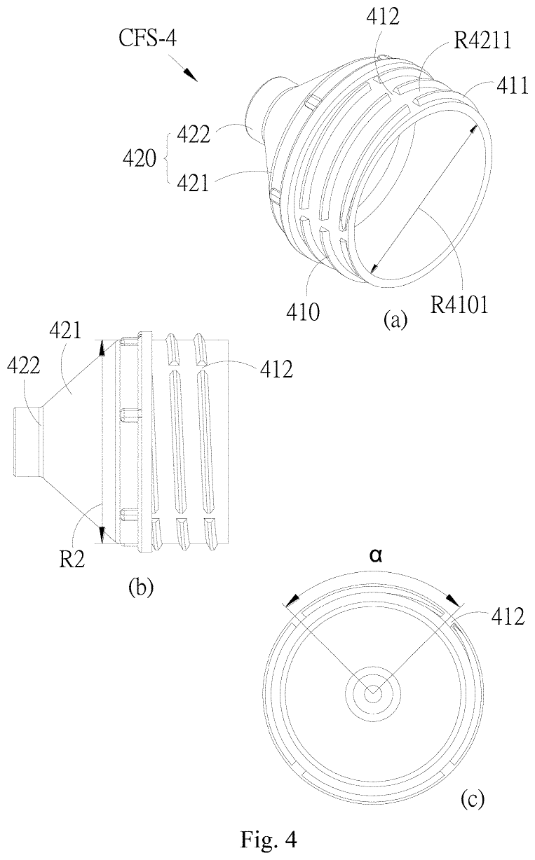

Refer to FIG. 4 for three structural diagrams of an embodiment of the filling hopper of the present invention, in which FIG. 4(a) to FIG. 4(c) are respectively a perspective view, a side view, and a top view of the filling hopper of the invention. The filling hopper CFS-4 may include a connecting member 410 and a hopper member 420.

The connecting member 410 may have a plurality of projections 411 provided on its outer surface. The connecting member 410 may have at least one vent hole 412. The at least one vent hole 412 may be provided in the outer surface of the connecting member 410 and penetrate the outer surface of the connecting member 410. The hopper member 420 may include a wide portion 421 and a narrow portion 422. The wide portion 421 is configured to connect with the connecting member 410. The narrow portion 422 is detachably connected to the first end portion 211. The hopper member 420 has a second inner diameter R2. The inner diameter R4101 of the connecting member 410 may be equal to the inner diameter R4211 of the wide portion 421. The inner diameter R4101 is preferably 20.9 mm˜32.9 mm, more preferably 22.9 mm˜30.9 mm, and even more preferably 24.9 mm˜28.9 mm. The inner diameter R4211 is preferably 23.5 mm˜35.5 mm, more preferably 25.5 mm˜33.5 mm, and even more preferably 27.5 mm˜31.5 mm. The second inner diameter R2 is gradually reduced from the wide portion 421 to the narrow portion 422, where the second inner diameter R2 is equal to the first inner diameter R1. In other words, the value of the second inner diameter R2 is variable, and the smallest value of the second inner diameter R2 may be equal to the first inner diameter R1. The second inner diameter R2 is preferably 23.5 mm˜35.5 mm, more preferably 25.5 mm˜33.5 mm, and even more preferably 27.5 mm˜31.5 mm.

As shown in FIG. 4(c), the at least one vent hole 412 may penetrate the connecting member 410. To prevent the received cartilage from leaking out, there may be one or a plurality of vent holes 412; that is to say, there may be one, two, three, or more vent holes 412 as needed. The vent holes 412 may be provided at equal intervals along the circumference of the connecting member 410, with each two adjacent vent holes 412 forming a central angle α with respect to the center of the cross section of the connecting member 410. For example, the angle α is 90 degrees when there are four equally spaced vent holes 412, and 45 degrees when there are eight equally spaced vent holes 412.

In one embodiment, the cartilage filling system CFS further includes a collection cup. The inner surface of the collection cup has structures corresponding in shape to the projections 411 of the connecting member 410 of the filling hopper CFS-4 to enable connection between the collection cup and the connecting member 410. A user who has put cartilage tissue into the collection cup and joined the collection cup to the filling hopper CFS-4 of the cartilage filling system CFS may flip the assembly over to allow the cartilage tissue in the collection cup to be received in the cartilage filling system CFS by way of gravity, and during the process, the vent holes 412 in the connecting member 410 will effectively release any trapped air so that the cartilage tissue can be transferred smoothly into the cartilage filling system CFS.

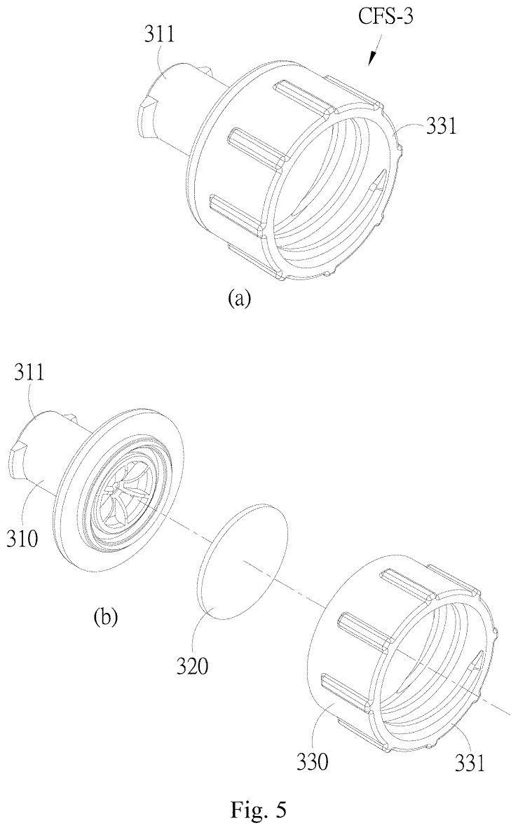

Refer to FIG. 5 for two structural diagrams of an embodiment of the filtering adapter of the present invention, in which FIG. 5(a) and FIG. 5(b) are respectively an assembled perspective view and an exploded perspective view of the filtering adapter of the invention. The filtering adapter CFS-3 may have a first adapter end 311 and a second adapter end 331. The second adapter end 331 is configured to connect with the second end portion 213 of the syringe 210. The first adapter end 311 is configured to connect with an external syringe, such as one with a Luer-taper connector.

As shown in FIG. 5(b), the filtering adapter CFS-3 may include a vaned supporting portion 310, a filter membrane 320, and a connecting member 330. The filter membrane 320 is detachably provided on the vanes of the vaned supporting portion 310 so that the liquids to be filtered out can flow out through the gaps between the vanes. The filter membrane 320 may be provided between the vaned supporting portion 310 and the connecting member 330. A user may use a filter membrane 320 of the desired dimensions and pore size in order to filter out unwanted liquids.

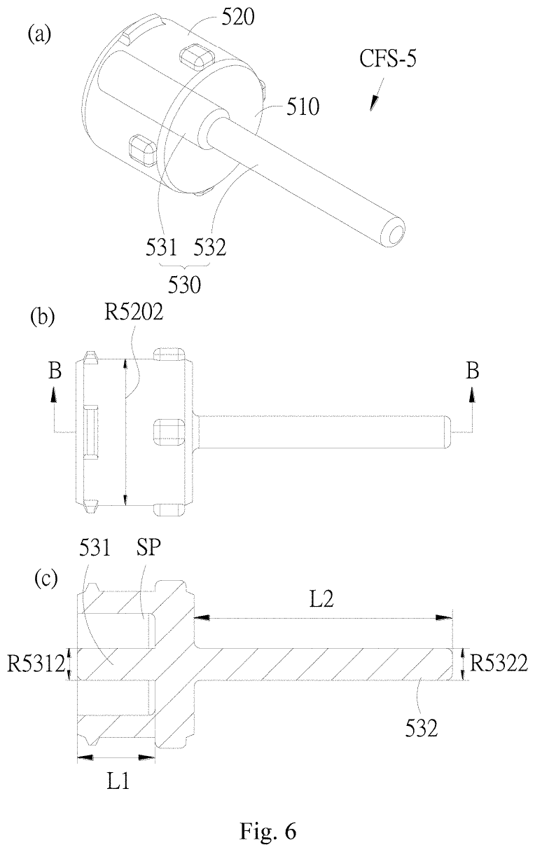

Continued from the above, the cartilage filling system of the present invention may further include a pushing member and a quantifying carrier. Refer to FIG. 6 for three structural diagrams of an embodiment of the pushing member of the invention, in which FIG. 6(a) to FIG. 6(c) are respectively a perspective view, a side view, and a line B-B sectional view of the pushing member of the invention.

The pushing member CFS-5 includes a bottom side 510, a sidewall 520, and a rod member 530. The sidewall 520 may surround the bottom side 510 and may be connected to the bottom side 510 such that the sidewall 520 and the bottom side 510 form a receiving space SP. The rod member 530 may include a first rod portion 531 and a second rod portion 532. The rod member 530 may penetrate the bottom side 510 such that the first rod portion 531 is in the receiving space SP while the second rod portion 532 is outside the receiving space SP.

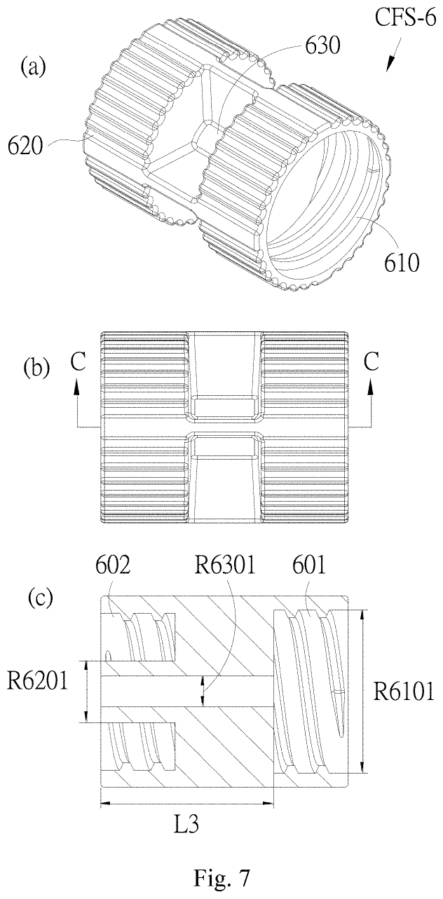

Refer to FIG. 7 for three structural diagrams of an embodiment of the quantifying carrier of the present invention, in which FIG. 7(a) to FIG. 7(c) are respectively a perspective view, a side view, and a line C-C sectional view of the quantifying carrier of the invention.

The quantifying carrier CFS-6 may include a first connecting portion 610, a second connecting portion 620, and a quantifying member 630. The inner diameter R6101 of the first connecting portion 610 may correspond to the outer diameter R2132 of the second end portion 213 of the syringe 210 to enable connection between the quantifying carrier CFS-6 and the syringe through the first connecting portion 610. The inner diameter R6201 of the second connecting portion 620 may correspond to the outer diameter R5202 of the sidewall 520 of the pushing member CFS-5 to enable connection between the quantifying carrier CFS-6 and the pushing member through the second connecting portion 620. The quantifying member 630 may be shaped as a hollow cylinder. One end of the quantifying member 630 is connected to the first connecting portion 610, and the other end of the quantifying member 630 is connected to the second connecting portion 620. The quantifying member 630 has a length L3. The outer diameter R5202 is preferably 5.3 mm˜17.3 mm, more preferably 7.3 mm˜15.3 mm, and even more preferably 9.3 mm˜13.3 mm. The inner diameter R6101 is preferably 7.3 mm˜19.3 mm, more preferably 9.3 mm˜17.3 mm, and even more preferably 11.3 mm˜15.3 mm. The inner diameter R6201 is preferably 2.5 mm˜5.5 mm, more preferably 3 mm˜5 mm, and even more preferably 3.5 mm˜4.5 mm.

In one embodiment, referring to FIG. 6(c) and FIG. 7(c), the first rod portion 531 has a length L1, which is preferably 5.4 mm˜6.6 mm, more preferably 5.6 mm˜6.4 mm, and even more preferably 5.8 mm˜6.2 mm; and the second rod portion 532 has a length L2, which is preferably 18.5 mm˜21.5 mm, more preferably 19 mm˜21 mm, and even more preferably 19.5 mm˜20.5 mm. In one embodiment, the length L1 of the first rod portion 531 is less than the length L2 of the second rod portion 532, and the length L3 of the quantifying member 630 of the quantifying carrier CFS-6 is greater than the length L1 of the first rod portion 531 and less than the length L2 of the second rod portion 532. The length L3 is preferably 12.5 mm˜15.5 mm, more preferably 13 mm˜15 mm, and even more preferably 13.5 mm˜14.5 mm.

In another embodiment, the inner diameter R6301 of the quantifying member 630 may be the same as the first inner diameter R1. The first rod portion 531 may have an outer diameter R5312, and the second rod portion 532 may have an outer diameter R5322. The outer diameter R5312 of the first rod portion 531 may be greater than or equal to the first inner diameter R1 of the syringe 210 such that a space of a fixed volume is formed when the pushing member CFS-5 is in a forwardly inserted state. The outer diameter R5322 of the second rod portion 532 may be equal to the inner diameter R6301 of the quantifying member 630 of the quantifying carrier CFS-6 so that the second rod portion 532 can be inserted into the quantifying member 630 and thereby bring the pushing member CFS-5 into the reversely inserted state. The inner diameter R6301 is preferably 1.9 mm˜2.9 mm, more preferably 2.1 mm˜2.9 mm, and even more preferably 2.3 mm˜2.7 mm. The outer diameter R5312 is preferably 1.9 mm˜2.9 mm, more preferably 2.1 mm˜2.9 mm, and even more preferably 2.3 mm˜2.7 mm. The outer diameter R5322 is preferably 1.9 mm˜2.9 mm, more preferably 2.1 mm˜2.9 mm, and even more preferably 2.3 mm˜2.7 mm.

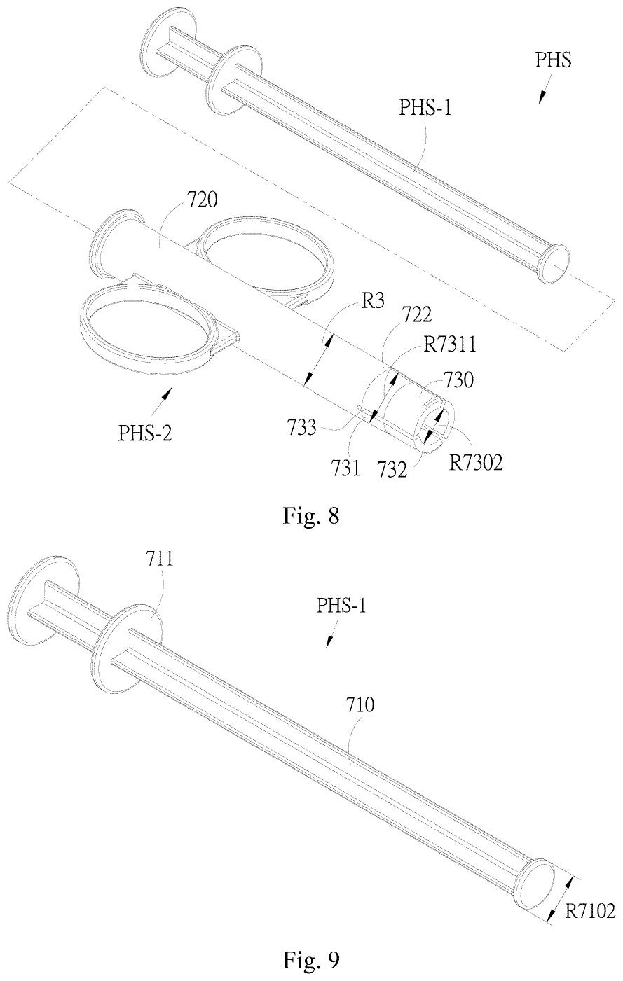

Continued from the above, the cartilage filling system of the present invention can be used in conjunction with a scaffold conveyor. Refer to FIG. 8 for a structural diagram of an embodiment of the scaffold conveyor of the invention. The scaffold conveyor PHS may include a conveying push-rod PHS-1 and a conveying member PHS-2.

Refer to FIG. 9 for a structural diagram of an embodiment of the conveying push-rod of the present invention. The conveying push-rod PHS-1 may include a conveying push-rod body 710 and a conveying push portion 711. The conveying push portion 711 may be provided at one end of the conveying push-rod body 710. Like the filling push portion 120, the conveying push portion 711 may correspond in shape to a user's finger. The conveying push-rod body 710 may have an outer diameter R7102.

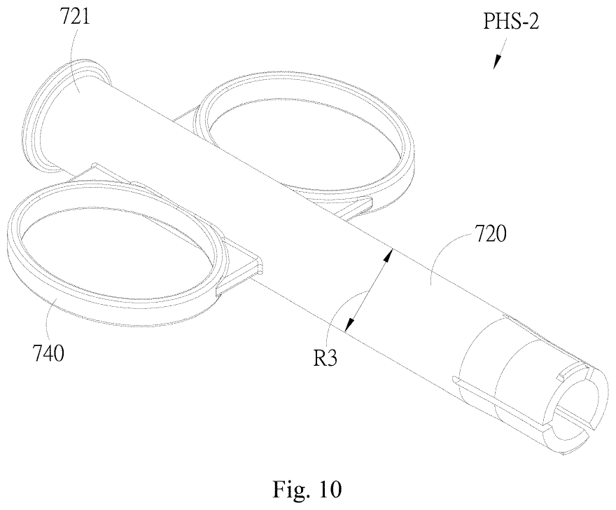

Refer to FIG. 10 for a structural diagram of an embodiment of the conveying member of the present invention. The conveying member PHS-2 may include a conveying tube 720, a grasping portion 730, and a conveying wing plate 740. The conveying tube 720 may have a conveying inner diameter R3. The conveying inner diameter R3 is preferably 6 mm˜9 mm, more preferably 7 mm˜9 mm, and even more preferably 8 mm˜9 mm. The conveying tube 720 may include a first conveying end 721 and a second conveying end 722. The conveying wing plate 740 may be provided on the outer surface of the conveying tube 720. The conveying wing plate 740 may be provided adjacent to the first conveying end 721. The shape of the conveying wing plate 740 may be similar to that of the grip portion 220. The grasping portion 730 may be a hollow circular base including a wide end 731 and a narrow end 732. The wide end 731 has a larger diameter than the narrow end 732. The peripheral surface of the hollow circular base is provided with a plurality of grooves 733 that are arranged at intervals. The grasping portion 730 is configured to connect with the conveying tube 720. The wide end 731 of the grasping portion 730 is configured to connect with the second conveying end 722 of the conveying tube 720. The wide end 731 has an inner diameter R7311, and the inner diameter R7311 is equal to the conveying inner diameter R3. The grooves 733 are provided along the extending direction of the grasping portion 730 and penetrate the grasping portion 730 such that the grasping portion 730 is able to grasp an object whose outer diameter ranges between the inner diameter of the wide end 731 and the inner diameter of the narrow end 732. The inner diameter R7311 is preferably 8 mm˜9.2 mm, more preferably 8.2 mm˜9 mm, and even more preferably 8.4 mm˜8.8 mm.

In one embodiment, the outer diameter R7102 of the conveying push-rod body 710 corresponds to the conveying inner diameter R3 so that at least a portion of the conveying push-rod body 710 can be inserted into the conveying tube 720. In addition, the inner diameter R7302 of the grasping portion 730 may correspond to the inner diameter R6101 of the first connecting portion 610 of the quantifying carrier CFS-6 so that the quantifying carrier CFS-6 can be mounted around the grasping portion 730. The outer diameter R7102 is preferably 8 mm˜8.8 mm, more preferably 8.2 mm˜8.8 mm, and even more preferably 8.4 mm˜8.8 mm. The inner diameter R7302 is preferably 6 mm˜8.5 mm, more preferably 7 mm˜8.5 mm, and even more preferably 8 mm˜8.5 mm.

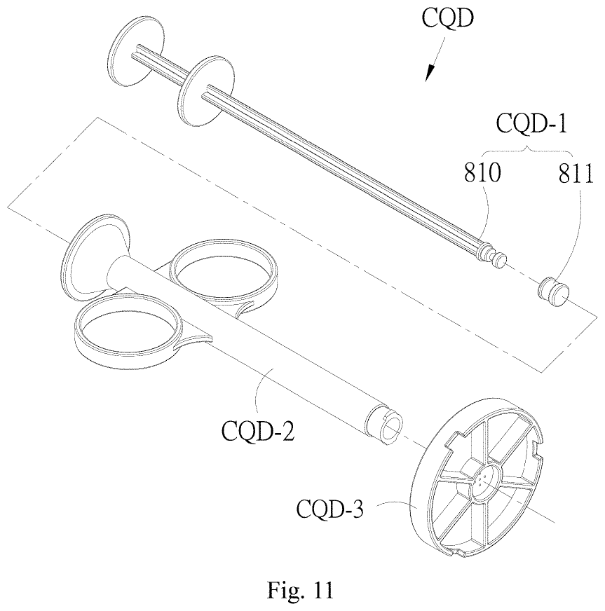

Refer to FIG. 11 for a structural diagram of an embodiment of the cartilage quantifying device of the present invention. The cartilage quantifying device CQD may include a quantifying push-rod CQD-1, a quantifying member CQD-2, and a quantifying base CQD-3. The quantifying member CQD-2 is configured to connect with the quantifying base CQD-3, and the quantifying push-rod CQD-1 is configured to be inserted into the quantifying member CQD-2.

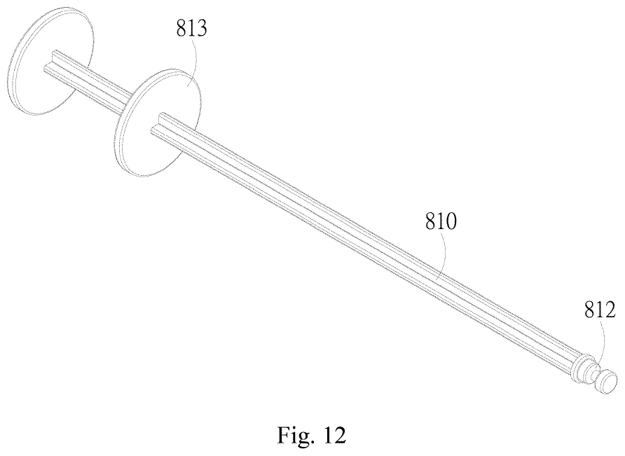

Refer to FIG. 12 for a structural diagram of an embodiment of the quantifying push-rod of the present invention. The quantifying push-rod CQD-1 may have a quantifying push-rod body 810 and a quantifying push portion 813. One end (hereinafter referred to as the first end) of the quantifying push-rod body 810 may be provided with a pointed end 812 that extends outward. The other end of the quantifying push-rod body 810 may be provided with the quantifying push portion 813. A user may push the quantifying push portion 813 and thereby push the quantifying push-rod body 810.

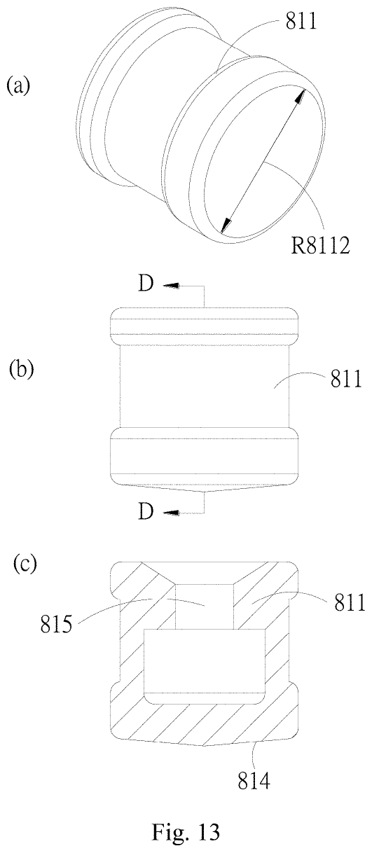

Refer to FIG. 13 for three structural diagrams of an embodiment of the quantifying plunger of the present invention, in which FIG. 13(a) to FIG. 13(c) are respectively a perspective view, a side view, and a line D-D sectional view of the quantifying plunger of the invention. The quantifying plunger 811 is configured to be mounted around the first end of the quantifying push-rod body 810. The quantifying plunger 811 may have a cavity 815 that corresponds in shape to the pointed end 812. The outer diameter R8112 of the quantifying plunger 811 is slightly greater than that of the quantifying push-rod body 810, and the quantifying plunger 811 is made of an elastic material such as silicone or rubber in order to not only increase the friction generated by pushing the quantifying push-rod body 810, but also ensure that the quantifying push-rod body 810 can be smoothly pushed, the objective being to push out the quantified cartilage completely. The outer diameter R8112 is preferably 4.4 mm˜5.6 mm, more preferably 4.6 mm˜5.4 mm, and even more preferably 4.8 mm˜5.2 mm.

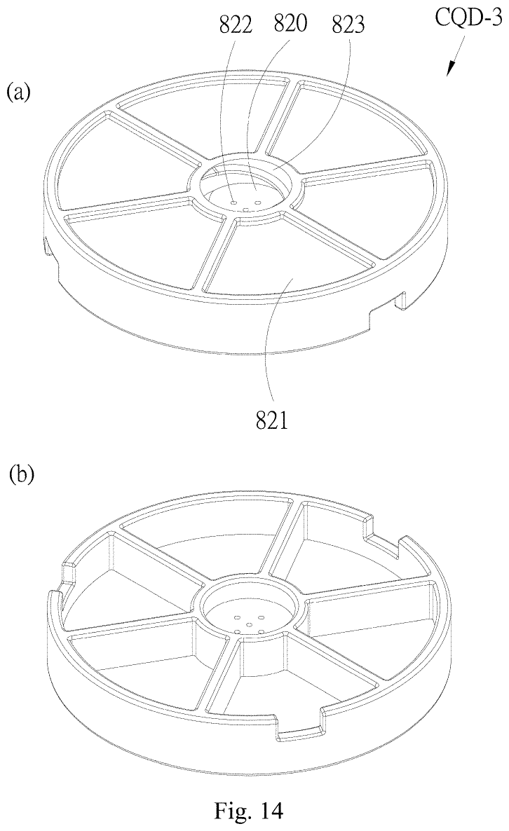

Refer to FIG. 14 for two structural diagrams of an embodiment of the quantifying base of the present invention, in which FIG. 14(a) and FIG. 14(b) are respectively a top view and a bottom view of the quantifying base of the invention. The quantifying base CQD-3 may be of any appropriate shape such as a regular or irregular polygonal shape or a circular shape. The quantifying base CQD-3 may include an upper connecting groove 820 and a plurality of supporting units 821. The supporting units 821 may be sequentially arranged around the upper connecting groove 820 and about a center of circle defined by the center of the upper connecting groove 820. The supporting units 821 may be connected to one another but are not necessarily so. Each of the supporting units 821 may be connected to the upper connecting groove 820.

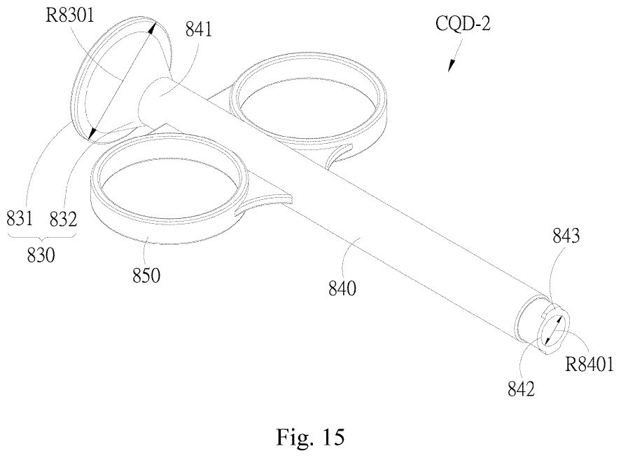

Refer to FIG. 15 for a structural diagram of an embodiment of the quantifying member of the present invention. The quantifying member CQD-2 may include a hopper portion 830, a quantifying tube 840, and a quantifying grip portion 850. The hopper portion 830 may have a wide end 831 and a narrow end 832. The quantifying tube 840 may be shaped as a hollow cylinder. The quantifying tube 840 has a first quantifying end 841 connected to the narrow end 832 of the hopper portion 830 and a second quantifying end 842 detachably connected to the upper connecting groove 820. The quantifying tube 840 may have an inner diameter R8401. The hopper portion 830 may have an inner diameter R8301. The inner diameter R8301 of the hopper portion 830 may be gradually reduced from the wide end 831 to the narrow end 832, where the inner diameter R8301 is equal to the inner diameter R8401. The quantifying grip portion 850 may be provided on the outer surface of the quantifying tube 840. The shape of the quantifying grip portion 850 may be similar to that of the grip portion 220. The outer diameter R8112 of the quantifying plunger 811 corresponds to the inner diameter R8401 of the quantifying tube 840, and at least a portion of the quantifying push-rod CQD-1 is insertable into the quantifying tube 840 such that a quantifying space is formed between the surface 814 of the quantifying plunger 811, the quantifying tube 840, and the upper connecting groove 820. The inner diameter R8301 is preferably 18.5 mm˜21.5 mm, more preferably 19 mm˜21 mm, and even more preferably 19.5 mm˜20.5 mm. The inner diameter R8401 is preferably 4.4 mm˜5.6 mm, more preferably 4.6 mm˜5.4 mm, and even more preferably 4.8 mm˜5.2 mm.

Referring also to FIG. 14(a), the second quantifying end 842 of the quantifying tube 840 has an engaging element 843, and the inner wall of the upper connecting groove 820 has a coupling portion 823 corresponding in shape to the engaging element 843 to enable detachable connection between the second quantifying end 842 of the quantifying tube 840 and the upper connecting groove 820. As shown in FIG. 14(b), the bottom side of the upper connecting groove 820 is penetrated by a plurality of vent holes 822. The vent holes 822 allow the air trapped by inserting at least a portion of the quantifying push-rod CQD-1 into the quantifying tube 840 to flow out. The quantifying tube 840 may have graduation lines. When at least a portion of the quantifying push-rod CQD-1 is inserted into the quantifying tube 840 and the surface 814 of the quantifying plunger 811 is aligned with one of the graduation lines, a quantifying space corresponding to the value of that graduation line is formed between the surface 814 of the quantifying plunger 811, the quantifying tube 840, and the upper connecting groove 820.

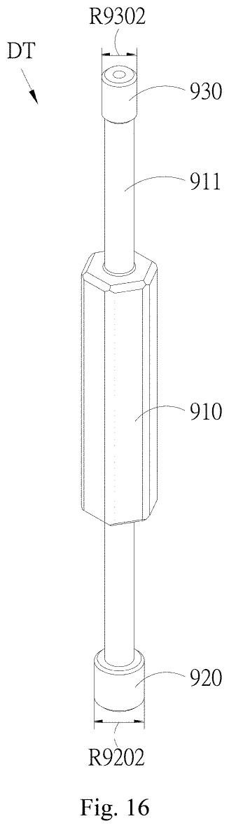

Refer to FIG. 16 for a structural diagram of an embodiment of the depth measuring device in the surgical instrument kit of the present invention. The depth measuring device DT is penetrated by a guide hole 911 along the extending direction of the depth measuring device DT so as to be guided with a K-wire. The depth measuring device DT may include a guiding portion 910, a positioning portion 920, and a measuring portion 930. The guiding portion 910 may be shaped as a rod and is configured to be gripped by a user. The positioning portion 920 may be provided at one end of the guiding portion 910. The positioning portion 920 may have a positioning outer diameter R9202. The positioning outer diameter R9202 is preferably 7.9˜9.1 mm, more preferably 8.1˜8.9 mm, and even more preferably 8.3˜8.7 mm. The measuring portion 930 may be provided at the other end of the guiding portion 910. The measuring portion 930 has a measuring outer diameter R9302, which is less than the positioning outer diameter R9202. The measuring outer diameter R9302 is preferably 5.4˜6.6 mm, more preferably 5.6˜6.4 mm, and even more preferably 5.8˜6.2 mm. In addition, the measuring outer diameter R9302 of the measuring portion 930 may correspond to the outer diameter of the scaffold to be implanted. The length of the measuring portion 930 may also correspond to the length of the scaffold to be implanted.

In another embodiment, the surgical instrument kit of the present invention may further include a bone drill.

The surgical instrument kit of the present invention may additionally include a toolbox. The toolbox may have receiving portions corresponding respectively to the cartilage filling system CFS, the cartilage quantifying device CQD, the scaffold conveyor PHS, and the depth measuring device DT so that the cartilage filling system CFS, the cartilage quantifying device CQD, the scaffold conveyor PHS, and the depth measuring device DT can be placed in the toolbox. The cartilage filling system CFS, the cartilage quantifying device CQD, and the scaffold conveyor PHS may be formed of medical-grade transparent plastic.

The present invention may be implemented with the first inner diameter R1 being 2.5 mm, the second inner diameter R2 being 29.5 mm, the conveying inner diameter R3 being 8.8 mm, the inner diameter R4101 being 26.9 mm, the inner diameter R4211 being 29.5 mm, the inner diameter R6101 being 13.2 mm, the inner diameter R6201 being 4 mm, the inner diameter R7311 being 8.6 mm, the inner diameter R8401 being 5 mm, the inner diameter R8301 being 20 mm, the outer diameter R2132 being 12.4 mm, the outer diameter R5202 being 11.3 mm, the outer diameter R5312 being 2.5 mm, the outer diameter R5322 being 2.5 mm, the inner diameter R7302 being 8.2 mm, the positioning outer diameter R9202 being 8.5 mm, and the measuring outer diameter R9302 being 6 mm, and can be used in conjunction with a Chondroplug scaffold with a diameter of 5.5 mm˜10.0 mm.

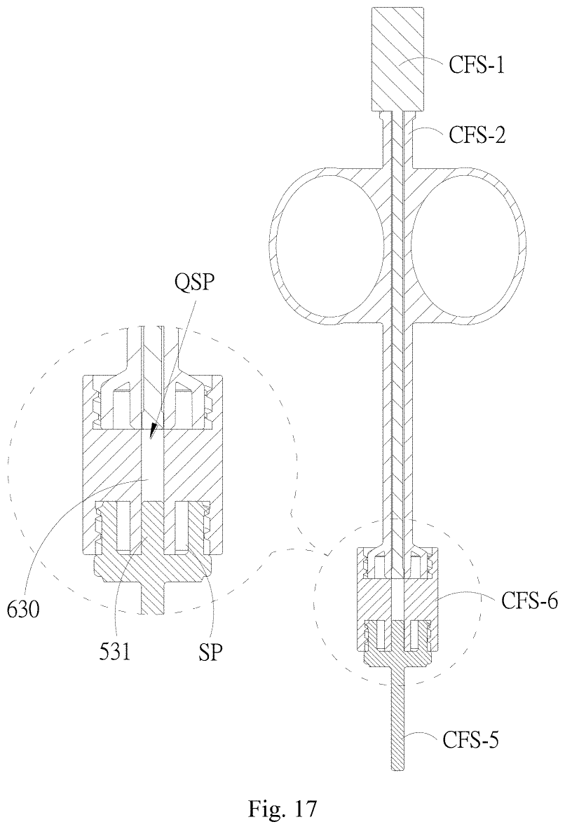

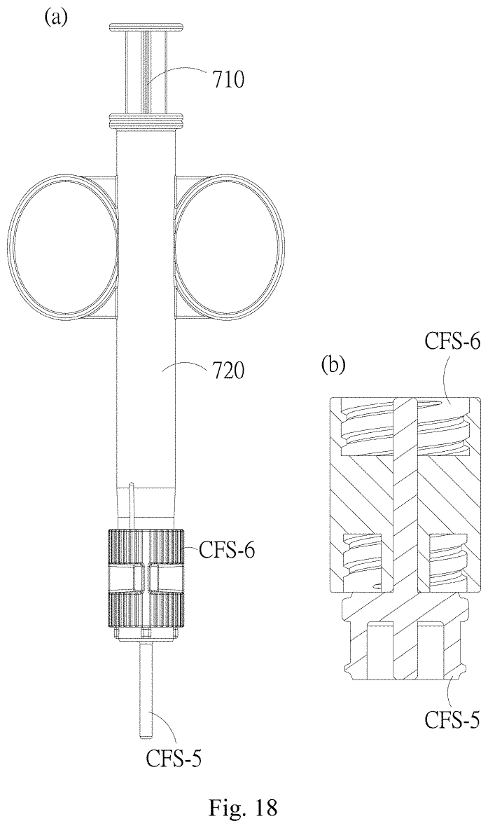

The methods of operating the cartilage filling system of the present invention and of operating the surgical instrument kit including the system are detailed below with reference to FIG. 17 and FIG. 18.

To start with, a block of cartilage is obtained from a non-weight-bearing site near the to-be-treated lesion with a scalpel and cut into 3 mm×3 mm cartilage chips. The cartilage chips are put into the quantifying member CQD-2 and the quantifying base CQD-3, and then the quantifying push-rod CQD-1 is pushed downward in order to quantify the cartilage chips according to the graduation lines of the quantifying member CQD-2. The quantified cartilage chips are subjected to a necessary medical treatment before being transferred into the collection cup.

The filling hopper CFS-4, the filling member CFS-2, and the filtering adapter CFS-3 are sequentially connected. The loaded collection cup is connected to the filling hopper CFS-4 and then inverted in order for the cartilage chips received in the collection cup to fall into the filling hopper CFS-4 while the vent holes 412 in the connecting member 410 allow any trapped air to exit. Next, a sterilized syringe is connected to the filtering adapter CFS-3, and the plunger of the sterilized syringe is moved back and forth to create a suction force that helps the filter membrane 320 to filter out the remaining liquids and unwanted impurities, leaving the cartilage chips in the filling member CFS-2. After that, the filtering adapter CFS-3, the filling hopper CFS-4, and the sterilized syringe are removed.

Refer to FIG. 17 for a drawing in which an embodiment of the pushing member of the present invention and an embodiment of the quantifying carrier of the invention are put together. The filling member CFS-2, the quantifying carrier CFS-6, and the pushing member CFS-5 are sequentially connected, and the filling push-rod CFS-1 is inserted into the filling member CFS-2 in order to push the cartilage chips into the quantifying space QSP formed by the quantifying member 630 of the quantifying carrier CFS-6 and the first rod portion 531 of the pushing member CFS-5. Please note that the state in which the first rod portion 531 is inserted into the quantifying member 630 is referred to herein as the forwardly inserted state. After that, the filling push-rod CFS-1 and the filling member CFS-2 are removed, and an assembly of the quantifying carrier CFS-6 and the pushing member CFS-5 is obtained, with the cartilage chips received in the assembly.

Refer to FIG. 18 for a drawing in which an embodiment of the pushing member of the present invention and an embodiment of the quantifying carrier of the invention are shown in two different assembled states. More specifically, FIG. 18(a) shows the pushing member CFS-5 in the forwardly inserted state while FIG. 18(b) shows the pushing member CFS-5 in the reversely inserted state. As a Chondroplug scaffold can be divided into a scaffold body, in which cartilage is received, and a lid, with which the scaffold body is sealed, the scaffold body to be used is placed into the grasping portion 730 of the conveying member PHS-2 with the scaffold opening facing outward, and then the quantifying carrier CFS-6 and the pushing member CFS-5 are locked to the conveying member PHS-2 as shown in FIG. 18(a). The pushing member CFS-5 is subsequently removed, flipped over, and inserted back into the quantifying carrier CFS-6 in order for the second rod portion 532 of the pushing member CFS-5 to push the cartilage chips into the scaffold body as shown in FIG. 18(b). Lastly, the pushing member CFS-5 and the quantifying carrier CFS-6 are removed, and the lid is fitted into the scaffold body. The cartilage-filled scaffold is now ready to be placed into the lesion through the conveying member PHS-2.

The present invention also provides a method for treating a cartilage defect, and the method includes the steps of: providing a cartilage quantifying device CQD as shown in FIG. 11, wherein the cartilage quantifying device CQD includes a quantifying push-rod CQD-1, a quantifying member CQD-2, and a quantifying base CQD-3, and the quantifying member CQD-2 includes a hopper portion 830 and a quantifying tube 840; placing cartilage into the hopper portion 830; pushing the cartilage into the quantifying tube 840 with the quantifying push-rod CQD-1 until a particular quantifying graduation line is reached; separating the quantifying base CQD-3 from the quantifying member CQD-2; mincing the cartilage in the quantifying tube 840; providing a cartilage filling system CFS as shown in FIG. 1, wherein the cartilage filling system CFS includes a filling push-rod CFS-1, a filling member CFS-2, a filtering adapter CFS-3, and a filling hopper CFS-4; placing the minced cartilage into the filling hopper CFS-4; creating a vacuum through the filtering adapter CFS-3 such that the minced cartilage enters the filling member CFS-2; separating the filtering adapter CFS-3 from the filling member CFS-2; providing a quantifying carrier CFS-6 and a pushing member CFS-5 as shown in FIG. 17, wherein the pushing member CFS-5 includes a first rod portion 531 and a second rod portion 532; coupling the quantifying carrier CFS-6 to the filling member CFS-2, with the first rod portion 531 and the quantifying carrier CFS-6 forming a quantifying space QSP; pushing the minced cartilage in the filling member CF S-2 into the quantifying space QSP with the filling push-rod CFS-1; separating the filling member CFS-2 from the quantifying carrier CFS-6, in which the minced cartilage is received; providing a scaffold conveyor PHS as shown in FIG. 8, wherein the scaffold conveyor PHS includes a conveying push-rod PHS-1 and a conveying member PHS-2, and the conveying member PHS-2 includes a conveying tube 720 and a grasping portion 730; placing a scaffold into the grasping portion 730; coupling the quantifying carrier CFS-6 to the scaffold; separating the pushing member CFS-5 from the quantifying carrier CFS-6; pushing the minced cartilage in the quantifying carrier CFS-6 into the scaffold with the second rod portion 532; separating the quantifying carrier CFS-6 from the scaffold, in which the minced cartilage is received; and pushing the scaffold by means of the conveying push-rod PHS-1 and the conveying tube 720 such that the scaffold, in which the minced cartilage is received, is separated from the grasping portion 730 and enters the cartilage defect being treated.

The present invention further provides a method for treating a cartilage defect that, in addition to the steps stated in the previous paragraph, includes the steps of: providing a depth measuring device DT as shown in FIG. 16, wherein the depth measuring device DT includes a positioning portion 920 and a measuring portion 930; and measuring the diameter of the cartilage defect with either the positioning portion 920 or the measuring portion 930 in order to determine whether the diameter of the cartilage defect is the same as the outer diameter of the positioning portion 920 or the outer diameter of the measuring portion 930.

The above descriptions are merely illustrative and not restrictive. Any equivalent modification or variation that does not depart from the spirit and scope of the present invention should be included in the scope of the claim.

What is claimed is:

1. A cartilage filling system, comprising:
a filling push-rod comprising a filling push-rod body and a filling push portion, wherein the filling push portion is provided at an end of the filling push-rod body;
a filling member comprising:
a syringe shaped as a hollow cylinder and comprising a first end portion and a second end portion, wherein the syringe has a first inner diameter; and
a grip portion provided on an outer surface of the syringe;
a filling hopper comprising:
a connecting member having an outer surface with a plurality of projections, wherein the outer surface of the connecting member is penetrated by a vent hole; and
a hopper member comprising a wide portion and a narrow portion, wherein the wide portion is connected to the connecting member, the wide portion has an inner diameter equal to an inner diameter of the connecting member, the narrow portion is detachably connected to the first end portion, the hopper member has a second inner diameter, and the second inner diameter is gradually reduced from the wide portion to the narrow portion, at which the second inner diameter is equal to the first inner diameter; and
a filtering adapter having an end connected to the second end portion and an opposite end connectable to an external syringe, wherein the filtering adapter comprises a vaned supporting portion and a filter membrane detachably provided on the vaned supporting portion.

2. The cartilage filling system of claim 1, further comprising:
a pushing member including:
a bottom side;
a sidewall surrounding the bottom side and connected to the bottom side such that the sidewall and the bottom side form a receiving space; and
a rod member including a first rod portion and a second rod portion connected to each other, wherein the rod member penetrates the bottom side such that the first rod portion is in the receiving space while the second rod portion is outside the receiving space; and
a quantifying carrier including:
a first connecting portion having an inner diameter corresponding to an outer diameter of the second end portion of the syringe;
a second connecting portion having an inner diameter corresponding to an outer diameter of the sidewall of the pushing member; and
a quantifying member shaped as a hollow cylinder and having an end connected to the first connecting portion and an opposite end connected to the second connecting portion;
wherein the quantifying carrier is connected with the syringe through the first connecting portion and is connected with the pushing member through the second connecting portion.

3. The cartilage filling system of claim 2, wherein the first rod portion has a length less than a length of the second rod portion, and the quantifying member has a length greater than the length of the first rod portion and less than the length of the second rod portion.

4. The cartilage filling system of claim 3, wherein the quantifying member has an inner diameter which is the same as the first inner diameter, the first rod portion has an outer diameter greater than or equal to the first inner diameter, and the second rod portion has an outer diameter equal to the inner diameter of the quantifying member.

5. The cartilage filling system of claim 1, wherein the grip portion includes two ring-shaped gripping parts, each of the ring-shaped gripping parts is provided on the outer surface of the syringe, the grip portion is provided adjacent to the first end portion, and closed circular areas in the ring-shaped gripping parts lies in the same plane.

6. The cartilage filling system of claim 1, further comprising a collection cup having an inner surface having structures corresponding in shape to the projections of the connecting member of the filling hopper to enable connection between the collection cup and the connecting member.

7. A surgical instrument kit comprising:
the cartilage filling system of claim 1,
a scaffold conveyor,
a cartilage quantifying device, and
a depth measuring device.

8. The surgical instrument kit of claim 7, wherein the scaffold conveyor includes:
a conveying push-rod; and
a conveying member including:
a conveying tube having a conveying inner diameter and including a first conveying end and a second conveying end;
a conveying wing plate provided on an outer surface of the conveying tube and adjacent to the first conveying end; and
a grasping portion being a hollow circular base including a wide end and a narrow end, wherein the hollow circular base has a peripheral surface provided with a plurality of grooves that are arranged at intervals, the wide end is configured to connect with the second conveying end, and the wide end has an inner diameter equal to the conveying inner diameter;
wherein the conveying push-rod has an outer diameter corresponding to the conveying inner diameter so that at least a portion of the conveying push-rod can be inserted into the conveying tube, and the grasping portion has an outer diameter corresponding to the inner diameter of the first connecting portion.

9. The surgical instrument kit of claim 7, wherein the cartilage quantifying device includes:
a quantifying push-rod having a quantifying push-rod body and a quantifying plunger mounted around an end of the quantifying push-rod body;
a quantifying base including a plurality of supporting units and an upper connecting groove, wherein the supporting units are sequentially arranged around the upper connecting groove and about a center of circle defined by the center of the upper connecting groove, the supporting units are connected to one another, and each of the supporting units is connected to the upper connecting groove; and
a quantifying member including:
a hopper portion having a wide end and a narrow end;
a quantifying tube shaped as a hollow cylinder, wherein the quantifying tube has a first quantifying end connected to the narrow end of the hopper portion and a second quantifying end detachably connected to the upper connecting groove, the quantifying tube has an inner diameter, the hopper portion has an inner diameter, and the inner diameter of the hopper portion is gradually reduced from the wide end to the narrow end, where the inner diameter of the hopper portion is equal to the inner diameter of the quantifying tube; and
a quantifying grip portion provided on an outer surface of the quantifying tube;
wherein the quantifying plunger has an outer diameter corresponding to the inner diameter of the quantifying tube, and at least a portion of the quantifying push-rod is insertable into the quantifying tube such that a quantifying space is formed between a surface of the quantifying plunger, the quantifying tube, and the upper connecting groove.

10. The surgical instrument kit of claim 9, wherein the second quantifying end of the quantifying tube has an engaging element, and an inner wall of the upper connecting groove has a coupling portion corresponding in shape to the engaging element to enable detachable connection between the second quantifying end of the quantifying tube and the upper connecting groove.

11. The surgical instrument kit of claim 10, wherein the upper connecting groove has a bottom side penetrated by a plurality of vent holes, and the vent holes allow the air trapped by inserting at least a portion of the quantifying push-rod into the quantifying tube to flow out.

12. The surgical instrument kit of claim 9, wherein the quantifying tube has graduation lines, wherein at least a portion of the quantifying push-rod is inserted into the quantifying tube and the surface of the quantifying plunger is aligned with one of the graduation lines so that a quantifying space corresponding to the value of that graduation line is formed between the surface of the quantifying plunger, the quantifying tube, and the upper connecting groove.

13. The surgical instrument kit of claim 7, wherein the depth measuring device is penetrated by a guide hole along an extending direction of the depth measuring device, and the depth measuring device includes:
a guiding portion shaped as a rod;
a positioning portion provided at a first end of the guiding portion and having a positioning outer diameter; and
a measuring portion provided at a second end of the guiding portion and having a measuring outer diameter that is less than the positioning outer diameter.

14. A method for treating a cartilage defect, including steps of:
providing a cartilage quantifying device including a quantifying push-rod, a quantifying member, and a quantifying base, wherein the quantifying member includes a hopper portion and a quantifying tube;
placing cartilage into the hopper portion;
pushing the cartilage into the quantifying tube with the quantifying push-rod until a particular quantifying graduation line is reached;
separating the quantifying base from the quantifying member;
mincing the cartilage in the quantifying tube;
providing a cartilage filling system including a filling push-rod, a filling member, a filtering adapter, and a filling hopper;
placing the minced cartilage into the filling hopper;
creating a vacuum through the filtering adapter such that the minced cartilage enters the filling member;
separating the filtering adapter from the filling member;
providing a quantifying carrier and a pushing member including a first rod portion and a second rod portion;
coupling the quantifying carrier to the filling member, with the first rod portion and the quantifying carrier forming a quantifying space;
pushing the minced cartilage in the filling member into the quantifying space with the filling push-rod;
separating the filling member from the quantifying carrier, in which the minced cartilage is received;
providing a scaffold conveyor including a conveying push-rod and a conveying member, wherein the conveying member includes a conveying tube and a grasping portion;
placing a scaffold into the grasping portion;
coupling the quantifying carrier to the scaffold;
separating the pushing member from the quantifying carrier;
pushing the minced cartilage in the quantifying carrier into the scaffold with the second rod portion;
separating the quantifying carrier from the scaffold, in which the minced cartilage is received; and
pushing the scaffold by means of the conveying push-rod and the conveying tube such that the scaffold, in which the minced cartilage is received, is separated from the grasping portion and enters a cartilage defect being treated.

15. The method for treating a cartilage defect of claim 14, further including steps of:
providing a depth measuring device including a positioning portion and a measuring portion; and
measuring a diameter of the cartilage defect with either the positioning portion or the measuring portion in order to determine whether the diameter of the cartilage defect is the same as an outer diameter of the positioning portion or an outer diameter of the measuring portion.
